(19) United States
(12) Patent Application Publication
(10) Pub. No.: US 2025/0195709 A1
(43) Pub. Date: Jun. 19, 2025

(54) AireScent Solutions

(71) Applicant: Chandler Todd Brice, Troy, AL (US)

(72) Inventor: Chandler Todd Brice, Troy, AL (US)

(21) Appl. No.: 18/539,127

(22) Filed: Dec. 13, 2023

(51) Int. Cl. A61L 9/12; B01D 46/00; B01D 46/52

(52) U.S. Cl. CPC A61L 9/12; B01D 46/0038; B01D 46/521; A61L 2209/14; B01D 2275/10; B01D 2279/50

(57) ABSTRACT

A scented air filter designed to improve indoor air quality while enhancing the ambiance of living and working spaces. In the manufacturing process, meticulous attention is paid to the selection and integration of materials to ensure the filter's efficacy and fragrance diffusion capabilities. High-quality materials are chosen for their filtration efficiency and durability. The base filter, typically composed of fine mesh or porous materials, is intricately layered with the scent mechanisms. Fragrance oils are carefully infused into the filter material or encapsulated within microcapsules dispersed throughout the layers. This process allows for a controlled release of scents over time, ensuring a consistent and pleasant aroma in the filtered air. The choice of fragrances is a significant aspect of the manufacturing process, with options ranging from soothing lavender to invigorating citrus blends, catering to diverse consumer preferences and indoor environments.

Product Description: The AireScent Solutions Scented Air Filter for All Air Conditioning Units is a pioneering advancement in air filtration technology. This innovative product combines pleating and scent infusion technologies to revolutionize indoor air quality and ambiance. Engineered to cater to various applications, it ensures exceptional particle capture efficiency through its advanced filtration materials and pleated structure. Additionally, the integration of scent infusion technology releases carefully curated fragrances into the environment, transforming any space into a more inviting and pleasant atmosphere. Designed to accommodate air conditioning units of all sizes, from residential to industrial settings, this versatile air filter offers seamless integration without requiring modifications.

Product Name: AireScent Solutions

Key Features:
1. High Filtration Efficiency: The combination of the pleated structure and advanced filtration materials provides exceptional particle capture efficiency. Our air filter effectively captures particles of varying sizes, contributing to a healthier indoor environment by reducing the presence of allergens and contaminants.
2. Scent Infusion Technology: The innovative integration of scent infusion technology sets our air filter apart. As air passes through the filter, a carefully curated fragrance is released into the environment, transforming your living or working space into a more inviting and pleasant atmosphere.
3. Versatile Size Compatibility: Engineered to accommodate a wide range of air conditioning units, spanning residential, commercial, and industrial settings. Our scented and pleated air filter is customizable across various dimensions, ensuring effortless integration into any HVAC system without necessitating alterations. This adaptability enables seamless replacement of standard air filters across all sizes, offering convenience and flexibility for diverse applications.

Upgrade your indoor air quality experience with our scented air filter. Whether at home or in a professional setting, this advanced filtration solution offers a perfect blend of health and comfort. Breathe easier while enjoying the subtle and refreshing aroma that accompanies clean, purified air. Welcome a new era of air filtration technology into your space with our state-of-the-art scented and pleated air filter.

Sizes: We want to cover every size air filter ranging from smaller ones that could fit in the car, to residential sized filters, all the way to huge resort type filters! Below is a list of every size in inches of the air filters.
10″×10″×1″, 10″×14″×1″, 10″×20″×1″, 10″×24″×1″, 12″×12″×1″, 12″×16″×1″, 12″×18″×1″, 12″×20″×1″, 12″×24″×1″, 12″×30″×1″, 12″×36″×1″, 14″×14″×1″, 14″×18″×1″, 14″×20″×1″, 14″×24″×1″, 14″×25″×1″, 14″×30″×1″, 15″×20″×1″, 16″×16″×1″, 16″×18″×1″, 16″×20″×1″, 16″×24″×1″, 16″×25″×1″, 16″×30″×1″, 18″×18″×1″, 18″×20″×1″, 18″×24″×1″, 18″×25″×1″, 20″×20″×1″, 20″×22″×1″, 20″×24″×1″, 20″×25″×1″, 20″×30″×1″, 20″×32″×1″, 20″×36″×1″, 22″×22″×1″, 22″×24″×1″, 22″×25″×1″, 24″×24″×1″, 24″×25″×1″, 24″×30″×1″, 24″×32″×1″, 24″×36″×1″, 10″×10″×2″, 10″×14″×2″, 10″×20″×2″, 10″×24″×2″, 12″×12″×2″, 12″×16″×2″, 12″×18″×2″, 12″×20″×2″, 12″×24″×2″, 12″×30″×2″, 12″×36″×2″, 14″×14″×2″, 14″×18″×2″, 14″×20″×2″, 14″×24″×2″, 14″×25″×2″, 14″×30″×2″, 15″×20″×2″, 16″×16″×2″, 16″×18″×2″, 16″×20″×2″, 16″×24″×2″, 16″×25″×2″, 16″×30″×2″, 18″×18″×2″, 18″×20″×2″, 18″×24″×2″, 18″×25″×2″, 20″×20″×2″, 20″×22″×2″, 20″×24″×2″, 20″×25″×2″, 20″×30″×2″, 20″×32″×2″, 20″×36″×2″, 22″×22″×2″, 22″×24″×2″, 22″×25″×2″, 24″×24″×2″, 24″×25″×2″, 24″×30″×2″, 24″×32″×2″, 24″×36″×2″, 10″×10″×3″, 10″×14″×3″, 10″×20″×3″, 10″×24″×3″, 12″×12″×3″, 12″×16″×3″, 12″×18″×3″, 12″×20″×3″, 12″×24″×3″, 12″×30″×3″, 12″×36″×3″, 14″×14″×3″, 14″×18″×3″, 14″×20″×3″, 14″×24″×3″, 14″×25″×3″, 14″×30″×3″, 15″×20″×3″, 16″×16″×3″, 16″×18″×3″, 16″×20″×3″, 16″×24″×3″, 16″×25″×3″, 16″×30″×3″, 18″×18″×3″, 18″×20″×3″, 18″×24″×3″, 18″×25″×3″, 20″×20″×3″, 20″×22″×3″, 20″×24″×3″, 20″×25″×3″, 20″×30″×3″, 20″×32″×3″, 20″×36″×3″, 22″×22″×3″, 22″×24″×3″, 22″×25″×3″, 24″×24″×3″, 24″×25″×3″, 24″×30″×3″, 24″×32″×3″, 24″×36″×3″, 10″×10″×4″, 10″×14″×4″, 10″×20″×4″, 10″×24″×4″, 12″×12″×4″, 12″×16″×4″, 12″×18″×4″, 12″×20″×4″, 12″×24″×4″, 12″×30″×4″, 12″×36″×4″, 14″×14″×4″, 14″×18″×4″, 14″×20″×4″, 14″×24″×4″, 14″×25″×4″, 14″×30″×4″, 15″×20″×4″, 16″×16″×4″, 16″×18″×4″, 16″×20″×4″, 16″×24″×4″, 16″×25″×4″, 16″×30″×4″, 18″×18″×4″, 18″×20″×4″, 18″×24″×4″, 18″×25″×4″, 20″×20″×4″, 20″×22″×4″, 20″×24″×4″, 20″×25″×4″, 20″×30″×4″, 20″×32″×4″, 20″×36″×4″, 22″×22″×4″, 22″×24″×4″, 22″×25″×4″, 24″×24″×4″, 24″×25″×4″, 24″×30″×4″, 24″×32″×4″, 24″×36″×4″, 10″×10″×5″, 10″×14″×5″, 10″×20″×5″, 10″×24″×5″, 12″×12″×5″, 12″×16″×5″, 12″×18″×5″, 12″×20″×5″, 12″×24″×5″, 12″×30″×5″, 12″×36″×5″, 14″×14″×5″, 14″×18″×5″, 14″×20″×5″, 14″×24″×5″, 14″×25″×5″, 14″×30″×5″, 15″×20″×5″, 16″×16″×5″, 16″×18″×5″, 16″×20″×5″, 16″×24″×5″, 16″×25″×5″, 16″×30″×5″, 18″×18″×5″, 18″×20″×5″, 18″×24″×5″, 18″×25″×5″, 20″×20″×5″, 20″×22″×5″, 20″×24″×5″, 20″×25″×5″, 20″×30″×5″, 20″×32″×5″, 20″×36″×5″, 22″×22″×5″, 22″×24″×5″, 22″×25″×5″, 24″×24″×5″, 24″×25″×5″, 24″×30″×5″, 24″×32″×5″, 24″×36″×5″.

Field of Endeavor: The present invention pertains to the field of air filtration for air conditioning units. Specifically, it relates to an improved air filter that combines both scent infusion and pleating technologies to enhance air quality within enclosed spaces.

Description of the Invention: The invention is directed towards a novel air filter designed for use in various air conditioning units, regardless of their size. This unique air filter employs a combination of two distinct features: scent infusion and pleating. The scent infusion aspect involves incorporating a fragrance or scent-capturing material within the filter medium. This allows the air filter to not only capture particulate matter but also disperse a pleasant scent into the filtered air, thereby improving the overall indoor air quality.

Additionally, the air filter employs a pleated structure, enhancing its filtration efficiency and surface area for trapping airborne particles. The pleating increases the filter's particle retention capacity, ensuring effective removal of dust, pollen, allergens, and other pollutants from the air. The filter's pleated design also prolongs its lifespan by reducing the frequency of replacements, providing a cost-effective and environmentally friendly solution. The invention caters to air conditioning units of all sizes, from small residential units to large commercial installations. This versatile approach ensures that indoor spaces of varying dimensions can benefit from improved air quality and a pleasant ambiance.

Relevant Prior Art and Known Information: In the existing prior art, air filters for air conditioning units are designed primarily for efficient particulate matter removal. Pleated air filters have been used to increase filtration efficiency, while some filters are infused with fragrances to address indoor odor issues. However, there remains a need for a comprehensive solution that combines both scent infusion and pleating technologies in a single air filter.

References to specific documents related to the invention and its components:
1. “Improving Indoor Air Quality through Scented Filters,” Air Quality Journal, Volume 17, Issue 3, Page 45-57, (2018).
2. “Enhancing Air Filter Efficiency using Pleating Techniques,” HVAC Engineering Conference Proceedings, (2019), Page 112-125.

Problems Addressed by the Invention: The existing air filtration technologies lack a unified approach to simultaneously improve air quality and offer a pleasant scent within indoor spaces. Conventional air filters often require frequent replacement due to their limited capacity, leading to increased costs and waste. The present invention addresses these problems by providing an air filter that combines scent infusion and pleating, thereby enhancing air quality, reducing odor, and extending the filter's lifespan.

By introducing a scented and pleated air filter suitable for all air conditioning unit sizes, this invention seeks to offer a comprehensive solution to the challenges posed by conventional filters in the prior art.

Summary: The present invention is a novel air filter designed for use in various air conditioning units, offering a combination of effective filtration, pleasant scent diffusion, and versatile sizing. Traditional air filters often lack scent capabilities and may not provide optimal filtration efficiency. The newly proposed air filter addresses these drawbacks by integrating scent-emitting technology and pleated filter design, catering to all sizes of air conditioning units.

### Advantages

1. Scent Dispersion: The integration of scent-emitting elements within the air filter enhances indoor air quality by releasing a pleasant fragrance. This not only improves the ambiance but also masks unpleasant odors often associated with air conditioning systems.
2. Versatile Sizing: The air filter is adaptable to various air conditioning unit sizes, ensuring compatibility across residential, commercial, and industrial applications. This eliminates the need for multiple filter designs, simplifying inventory management.
3. Addressing Prior Issues: Conventional filters often neglect the importance of scent diffusion and focus solely on filtration. The new invention addresses this gap by incorporating scent technology alongside effective filtration, enhancing the overall indoor environment.

Object of the Invention: The primary objective of the present invention is to provide an improved air filter that combines efficient filtration with aromatic benefits, catering to all sizes of air conditioning units. By addressing the shortcomings of conventional filters and enhancing the user experience, the invention seeks to offer a versatile solution that enhances indoor air quality, ambiance, and overall comfort.

In conclusion, the proposed scented and pleated air filter for all air conditioning units represents an innovative advancement in the field of air filtration technology. By concurrently addressing filtration efficiency, scent dispersion, and compatibility across various air conditioning unit sizes, the invention offers a comprehensive solution that enhances indoor air quality while creating a more pleasant indoor environment.

Detailed Description of the Invention: The present invention relates to an improved air filter for air conditioning units, designed to combine efficient air filtration with a pleasing scent. This description outlines the unique features of the scented and pleated air filter, its method of manufacture, and its utilization in air conditioning units of various sizes. The invention aims to provide enhanced air quality and a pleasant ambiance in indoor environments.

Background: Air conditioning systems play a crucial role in maintaining indoor comfort by regulating temperature and humidity. However, they often fall short in providing optimal indoor air quality. Traditional air filters primarily focus on particle removal and neglect the olfactory aspect. Additionally, existing scented solutions for indoor spaces lack the integration of air filtration. The current invention addresses these shortcomings by introducing a scented and pleated air filter for air conditioning units.

Description of the Invention: The scented and pleated air filter is an innovative combination of efficient air filtration and a pleasant scent diffusing system. It is suitable for use in air conditioning units of various sizes. The invention's components, structure, manufacturing process, and method of operation are detailed below.

### Components:

1. Filter Medium: The heart of the invention is a pleated air filter composed of a high-quality filter medium with excellent particulate removal capabilities. This filter medium can include materials like fiberglass, synthetic fibers, or other materials commonly used in air filters.
2. Scent Infusion System: Incorporated within the filter medium is a scent infusion system. This system consists of fragrance reservoirs or compartments containing scented substances, typically in liquid or gel form, designed to release fragrance into the filtered air stream.
3. Frame and Mounting Mechanism: The scented and pleated air filter is housed within a sturdy frame made of plastic or cardboard. The frame includes a secure mounting mechanism compatible with different air conditioning unit sizes.

### Manufacturing Process:

1. Preparation of Filter Medium: The filter medium material is selected based on its filtration efficiency. It is then pleated using industry-standard techniques to increase the available surface area for air filtration.
2. Integration of Scent Infusion System: During the pleating process, fragrance reservoirs or compartments are strategically positioned within the filter medium folds. These compartments are filled with scented substances that are designed to gradually release fragrance as air passes through the filter.
3. Assembly: The scented and pleated filter medium is carefully placed within the frame, ensuring proper alignment and secure mounting. The frame is constructed to prevent air bypass, ensuring that all air drawn into the air conditioning unit passes through the scented filter. This consists of a mesh covering for the filter medium material, as well as cardboard cross sections and all cardboard frame sections.

### Method of Use:

1. The scented and pleated air filter is selected based on the size compatibility with the air conditioning unit.
2. The existing air filter in the air conditioning unit is removed.
3. The scented and pleated air filter is inserted into the designated filter compartment of the air conditioning unit, following the manufacturer's instructions.
4. As air circulates through the air conditioning system, it passes through the scented filter medium. The scent infusion system releases fragrance into the filtered air, creating a pleasant aroma within the indoor environment.
5. The scented filter performs its primary function of efficiently removing particles from the air, thereby enhancing indoor air quality.

### Distinguishing Features:

1. Integration of Filtration and Scent: The scented air filter uniquely combines effective air filtration with the diffusion of a pleasant scent, improving both air quality and ambiance.
2. Wide Size Compatibility: The invention is adaptable to air conditioning units of various sizes, providing versatility to accommodate different systems.

Conclusion: The scented and pleated air filter for air conditioning units represents a significant advancement in indoor air quality and comfort. By integrating efficient filtration with a scented experience, the invention enhances both the health and sensory aspects of indoor environments. The detailed description above provides all necessary information for a person of ordinary skill in the relevant art to make, use, and understand the invention without undue experimentation. This description and the accompanying drawings fulfill the requirements of US patent regulations and enable the protection of this innovative solution.

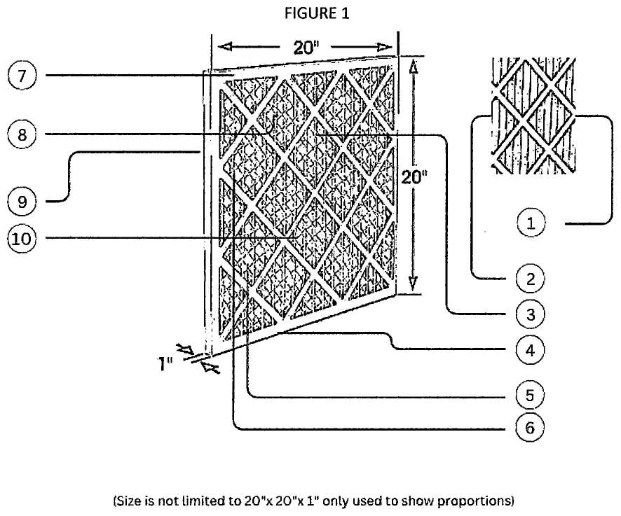

### All Numbers Listed Below are a Direct Reference to FIG. 1

1. Structural Encasement for Filtration Apparatus: A cross-sectional arrangement comprising a robust cardboard layer designed to encapsulate and fortify a mesh structure and filter medium material.
2. Enhanced Filtration Medium with Fragrance Reservoir Integration: A filter medium characterized by pleats enveloping fragrance reservoirs, thereby providing an advanced filtration system.
3. Mesh Integration System for Filtration Medium: A mesh overlay meticulously affixed to a pleated filter medium, ensuring a secure and durable integration.
4. Base Support Frame for Multi-component Filtration Device: A bottom cardboard frame meticulously designed to secure and stabilize all components within the filtration apparatus.
5. Efficient Filtration Pleats with Integrated Medium: A pleated filter medium distinguished by its structure that seamlessly incorporates optimal filtration efficiency.
6. Fragrance Reservoirs for Enhanced Aromatic Output: A fragrance reservoir system engineered to produce distinctive scents, strategically aligned for heightened efficiency within the filtration apparatus.
7. Top Support Frame for Comprehensive Filtration Assembly: An upper cardboard frame meticulously crafted to secure and unify all constituent elements of the filtration device.
8. Mesh Crossties for Reinforced Integration: Strengthening ties formed from mesh material, employed to secure the mesh to the filter medium, thereby fortifying the overall structure.
9. Side Support Frame for Holistic Component Stability: A lateral cardboard frame intricately designed to secure and harmonize all elements of the filtration device.
10. High-Performance Filtration Medium: A filter medium recognized for its ability to deliver superior air filtration, ensuring the provision of high-quality air purification.

1: A scent-infused air filter system designed to elevate indoor environments through a harmonious blend of air purification and fragrance diffusion. This system features a multi-layered filter structure equipped with scent infusion technology, allowing for the controlled release of captivating fragrances into the filtered air. The improvement in this claim lies in the innovative scent release mechanism, which enhances ambiance by providing a refreshing olfactory experience tailored to individual preferences. With this technology, the air filter not only captures particulate matter but also enriches indoor spaces with delightful scents, promoting a sense of comfort and well-being.

2: A high-efficiency scented air filter system engineered to provide exceptional air purification performance. This system comprises a multi-layered filter structure meticulously designed to maximize filtration efficiency, effectively capturing dust, pollen, and other airborne particles. The improvement in this claim focuses solely on optimizing filtration materials and design, ensuring thorough air purification without compromise. By leveraging advanced filtration technology, this system delivers superior filtration efficiency, promoting cleaner and healthier indoor air environments.

3: A versatile scented air filter compatible with various air conditioning units. The filter comprises a multi-layered structure adaptable to different sizes of air conditioning units. Integrated within the filter system is scent infusion technology, wherein the improvement comprises versatile size compatibility ensuring seamless replacement of standard air filters across residential, commercial, and industrial HVAC systems without requiring modifications. This compatibility feature enhances convenience and flexibility for diverse applications.
